(19) United States
(12) Patent Application Publication
(10) Pub. No.: US 2004/0254095 A1
(43) Pub. Date: Dec. 16, 2004

(54) Supplement for treating musculoskeletal disorders

(76) Inventors: Kenneth A. Martin, Maumelle, AR (US); Teresa Leigh Barr, Port Townsend, WA (US)

Correspondence Address: BUSKOP LAW GROUP, P.C., 1717 ST. JAMES PLACE, SUITE 500, HOUSTON, TX, 77056, US

(21) Appl. No.: 10/725,608

(22) Filed: Dec. 2, 2003

Related U.S. Application Data
(63) Continuation-in-part of application No. 10/241,542, filed on Sep. 11, 2002, now Pat. No. 6,660,308.

(51) Int. Cl. A61K 38/00; A61K 31/7008; A61K 31/714; A61K 31/525; A61K 31/51; A61K 31/355; A61K 31/225

(52) U.S. Cl. 514/2; 514/46; 514/62; 514/251; 514/300; 514/356; 514/276; 514/474; 514/458; 514/547; 514/52; 514/54

(57) ABSTRACT

The ingestible supplement for treating muskuloskeletal disorders is made of per supplement from about 250 mg to about 2500 mg of 2-amino-2-deoxyglucose sulfate, 2-amino-2-deoxyglucose sulfate hydrochloride, n-acetyl 2-amino-2-deoxyglucose sulfate, or combinations thereof; from about 40 mg to about 750 mg of acetyl myristoleate; from about 40 mg to about 800 mg of s-adenosylmethionine; from about 200 mg to about 2000 mg of a protein; from about 100 mg to about 2500 mg of Vitamin B, Vitamin C, Vitamin E, or complexes thereof; and from about 1000 mg to about 9000 mg of a fiber. A method for improving joint mobility in a subject comprising administering to the subject an amount of the supplement involves administering to a subject an amount of the supplement on a regular basis.

The present application claims priority to co-pending U.S. patent application Ser. No. 10/241,542 filed on Sep. 11, 2002.

### FIELD

Embodiments relate to a supplement used to treat musculoskeletal disorders.

### BACKGROUND

A need has existed for a large convenient dosage of glucosamine to be taken in one daily dose that can be quickly absorbed into the bloodstream. The large convenient dosage of glucosamine would thereby bypass the gut and eliminate the adverse reactions of the supplement's elemental ingredients as well as protect and buffer the lining of the stomach from the high dosages of the supplement's elemental ingredients. The large convenient dosage of glucosamine would also buffer the glucose levels in the blood and significantly reduce or eliminate the possible adverse effects of the supplement's essential ingredients.

The large convenient dosage of glucosamine would also make it possible to administer a one time daily large dose of the supplement that is fast absorbing, uses a powerful vasodilatation system, is tasteless in most liquids, odorless, non-steroidal, has no adverse symptoms of nausea, heartburn, diarrhea, constipation or headache as well as perfusing underperfused tissue by saturating the tissue, increases mobility of a mammal in all directions, decreasing inflammation, maintaining cartilage viability, increasing strength, muscle flexibility and health of tendons and ligaments, as well as increasing endurance and performance, and is also cost effective and capable of mass production.

Petrus U.S. Pat. No. 6,399,093 discloses a method and composition for the treatment of musculoskeletal disorders in mammals by the application of a topical composition comprising a permeation enhancing amount of one or more penetration enhancers, and one or more bio-affecting agents to provide anti-inflammatory relief and analgesia to the applied body part.

A need has existed for an ingestible supplement, which is fast absorbing, non-steroidal, and a vasodilator. A need has existed for a supplement, which is a one-time daily large dose, while avoiding the symptoms of nausea, heartburn, constipation, diarrhea, and headaches.

### SUMMARY

The ingestible supplement for treating muskuloskeletal disorders is made, per supplement, of from about 250 mg to about 2500 mg of 2-amino-2-deoxyglucose sulfate, 2-amino-2-deoxyglucose sulfate hydrochloride, n-acetyl 2-amino-2-deoxyglucose sulfate, and combinations thereof; from about 40 mg to about 750 mg of cetyl myristoleate; from about 40 mg to about 800 mg of s-adenosylmethionine; from about 200 mg to about 2000 mg of a protein; from about 100 mg to about 2500 mg of Vitamin B, Vitamin C, Vitamin E and complexes thereof; and from about 1000 mg to about 9000 mg of a fiber.

The method for improving joint mobility in a subject comprising administering to the subject an amount of the supplement involves administering to a subject an amount of the supplement on a regular basis.

### DETAILED DESCRIPTION OF THE PREFERRED EMBODIMENTS

Before explaining the present composition in detail, it is to be understood that the composition is not limited to the particular embodiments and that it can be practiced or carried out in various ways.

The ingestible supplement is usable for treating an inflammatory tissue or musculoskeletal disorders in a mammal involving tissue that is underperfused tissue, inflamed joints, or inflamed muscles, tendons and ligaments. The dosage amount is made from a glucosamine sulfate, a glucosamine hydrochloride, and an n-acetyl glucosamine and combinations thereof. Once daily dosages of the supplement are administered for about 10 to about 36 consecutive days, the user shows positive results regarding the treatment of musculoskeletal disorders.

The ingestible supplement is beneficial because the supplement perfuses underperufsed tissues. The supplement saturates the tissue, increases mobility in all directions, decreases inflammation, maintains cartilage viability, increases strength, increases muscle flexibility, and increases endurance.

The supplement uses an ingestible amount of glucosamine for treating an inflammatory tissue or musculoskeletal disorders in a mammal involving tissue that is underperfused tissue, inflamed joints or inflamed muscles and tendons. The dosage is a glucosamine sulfate, glucosamine hydrochloride, and an n-acetyl glucosamine and combinations thereof.

The ingestible supplement for treating muskuloskeletal disorders is made of from about 250 mg to about 2500 mg of 2-amino-2-deoxyglucose sulfate, 2-amino-2-deoxyglucose sulfate hydrochloride, n-acetyl 2-amino-2-deoxyglucose sulfate, or combinations thereof; from about 40 mg to about 750 mg of acetyl myristoleate; from about 40 mg to about 800 mg of s-adenosylmethionin; from about 200 mg to about 2000 mg of protein; from about 100 mg to about 2500 mg of a vitamin; and from about 1000 mg to about 9000 mg of a fiber. The vitamin can be Vitamin B, Vitamin C, Vitamin E, or complexes thereof. The amounts are per supplement.

Another embodiment is a method for improving joint mobility in a subject by administering to the subject an amount of the supplement.

The ingestible amount of glucosamine is used for treating an inflammatory tissue or musculoskeletal disorders in a mammal involving tissue that is underperfused tissue, inflamed joints or inflamed muscles and tendons. The dosage is a glucosamine sulfate, glucosamine hydrochloride, and an n-acetyl glucosamine and combinations thereof.

Glucosamine, whose scientific name is 2-amino-2-deoxyglucose sulfate, occurs naturally in the human body. The preferred range for the glucosamine is from about 250 mg to about 2500 mg. The preferred glucosamines are 2-amino-2-deoxyglucose sulfate; 2-amino-2-deoxyglucose sulfate hydrochloride; n-acetyl 2-amino-2-deoxyglucose sulfate; and combinations thereof. In a preferred embodiment, about 1200 mg to about 1500 mg by weight of 2-amino-2-deoxyglucose sulfate; 2-amino-2-deoxyglucose sulfate hydrochloride; n-acetyl 2-amino-2-deoxyglucose sulfate; or combinations thereof can be used.

Glucosamine provides strength, flexibility, and elasticity to cartilage and connective tissue by stimulating the production of glycosaminoglycans. Glucosamine also decreases inflammation that can lead to joint destruction. Glucosamine is involved in the formation of nails, tendons, skin, eyes, bones, ligaments, and heart valves. More importantly, glucosamine contributes to the strength and integrity of joint structures. Connective tissue and cartilage naturally contain high concentrations of glucosamine. When sufficient levels of glucosamine are present, cartilage retains its ability to hold water and act as a shock absorber. Glucosamine sulfate is a simple molecule composed of glucose, an amine, and sulfur. The joints are naturally rich in sulfur molecules, which form important cross-linkages with other molecules. These cross-linkages provide cartilage with its strength, structure, and shock-absorbing qualities. The strength, structure, and shock-absorbing qualities along with glucosamine's absorbability quality makes glucosamine sulfate the preferred form of supplemental glucosamine.

Each person produces a certain amount of glucosamine. As people age, the body no longer produces enough glucosamine causing musculoskeletal disorders such as arthritic conditions, deformed joints and limited joint and tendon movement. Numerous double-blind, placebo controlled glucosamine studies have been published, all reporting that glucosamine was indeed very effective in treating osteoarthritis and reducing inflammation and that its use is long-term safe. The studies have also shown that glucosamine provided in liquid form is absorbed more quickly, much more fully, and provides greater and longer lasting relief.

Methylsulfonylmethane (MSM) is a natural form of organic sulfur found in all living organisms. MSM is prevalent throughout the human body. MSM is an important food that plays many roles in the body, including the stimulation of the growth of healthy skin, hair and nails. MSM is needed by the body for healthy, connective tissues and joint function, proper enzyme activity and hormone balance, along with the proper function of the immune system. MSM is highly soluble in both oil and water.

As oxygen is transported from the lungs to the mitochondria, oxygen goes through a number of stages with continually decreasing oxidation potential or effective oxygen concentration. MSM easily and rapidly diffuses through the hydrophilic cell cytoplasm as well as the hydrophobic cell membranes. MSM has no barriers. The human body has no other molecules naturally occurring in our bodies similar to MSM. Oxygen transport is handled by passing it between different molecules that are hydrophilic in the cytoplasm and hydrophobic in the cell membranes

The preferred weight percent of MSM useable in the ingestible supplement is from about 40 mg to about 2500 mg.

Cetyl myristoleate has the chemical formula of cis-9-cetyl myristoleate and is a non-toxic substance found naturally in nuts, vegetables and some species of animals. The most preferable form of cetyl myristoleate and most preferred useable with the present supplement are derived from the vegetable oil myristoleic acid, which yields several fatty acid esters. Fatty acids in an ester form are highly resistant to oxidation both inside and outside the human body.

Cetyl myristoleate works as an anti-inflammatory by modulating the prostaglandin balance as an immune system modulator by inhibiting leukotriene, thereby reducing auto-immune responses. The supplement contains from about 40 mg to about 750 mg, preferably about 100 mg.

The supplement contemplates variations in the dosage amounts.

The supplement preferably has from about 200 mg to about 2000 mg of protein. Examples of proteins contemplated are whey proteins, soy or vegetable protein, urea, caseins and calcium caseinate, legume proteins, blue green micro algae proteins, nut and seed proteins and fish and animal proteins derived from eggs, meat or milk and combinations thereof. Blue-green micro-algae form spiral filaments or chains that as a dietary supplement that contain 65% protein in the dried state.

Various vitamins can be used. The supplement can include from about 100 mg to about 2500 mg of Vitamin B, Vitamin C, Vitamin E, zinc oxide, copper gluconate and potassium, or complexes of these vitamins.

If Vitamin B is used, the preferred dosage is from about 10 mg to about 500 mg Vitamin B. The preferred Vitamin B is a Vitamin B complex, The Vitamin B complex includes thiamine (Vitamin B1), riboflavin (Vitamin B2), niacin (Vitamin B3), pyridoxine (Vitamin B6), folic acid (Vitamin B9), cyanocobalamin (Vitamin B12), pantothenic acid, and biotin. The Vitamin B family aid metabolic activity and also produces energy. The Vitamin B family is also involved in making red blood cells that carry oxygen throughout the body and is necessary for every part of the body to work properly.

If Vitamin C is used, the preferred dosage is from about 250 mg to about 2,500 mg. Vitamin C comes in two basic forms: ascorbic acid and calcium ascorbates. Vitamin C is naturally found in citrus fruits, such as oranges, grapefruit, lemons, mangos, and the like, and in many green vegetables, such as asparagus, broccoli, spinach, green peppers, and peas, tomatoes, potatoes and cabbage. Ascorbic acid is the standard form of vitamin C. Examples of Vitamin C usable in the invention are ascorbic acid, mineral ascorbates, calcium ascorbates, a potassium ascorbate with at least one threonate and combinations of these.

Vitamin C is one of several antioxidants and maintains collagen, a protein necessary for the formation of skin, ligaments and bones. Vitamin C also enhances the immune systems that helps heal wounds and mend fractures. Vitamin C also aids in resisting some types of bacterial and viral infections, as well as also aiding in the absorption of iron.

If Vitamin E is used, the preferred dosage is from about 50 mg to about 5000 mg. Vitamin E is an antioxidant that protects cell membranes and other fat-soluble parts of the body. Vitamin E also plays a role in the body's ability to process glucose. In the last ten years, studies have clarified the function of Vitamin E in the cells. In addition to its antioxidant functions, Vitamin E is now known to act through other mechanisms, including direct effects on inflammation, blood cell regulation, connective tissue growth, and genetic control of cell division, improve circulation, and allow the muscles to use oxygen. Vitamin E is found naturally in wheat germ oil, nuts and seeds, whole grains, egg yolks, and leafy green vegetables and certain vegetable oils. The names of all types of vitamin E begin with either d or dl, which refer to differences in chemical structure. The d form is natural and also known as RRR-alpha tocopherol and dl is a synthetic version, more correctly known as all-rac-alpha tocopherol. The natural form is more active and better absorbed by the body. Vitamin E is traditionally measured in international units (IU). 100 IU of Vitamin E requires about 67 mg of the natural form, but closer to 100 mg of the synthetic form.

The supplement uses 67 mg for 100 IU of the preferable natural Vitamin E. The Vitamin E can be a tocopherol or tocopheryl followed by the name of what is attached to it, such as tocopheryl acetate. The most common forms of vitamin E are d-alpha tocopherol and d-alpha tocopheryl acetate or succinate and combinations or complexes thereof. The preferred Vitamin E is d-alpha tocopherol at 590 mg per dosage bar.

If Vitamin A is used, the preferred dosage is from about 250 mg to about 15,000 mg of Vitamin A. Vitamin A is fat-soluble vitamin. Retinol is one of the most active and usable forms of Vitamin A and is found in animal products, such as liver and eggs. Pro-vitamin A carotenoids are found in plant foods that contain darkly colored pigments that are converted to Vitamin A. Approximately 26% to 34% of Vitamin A consumed by men and women in the United States is provided by pro-vitamin A carotenoids. Beta-carotene is a pro-vitamin A carotenoid that is more efficiently converted to retinol than other carotenoids. The preferred Vitamin A is beta-carotene.

Omega 3 fatty acids also aid in the prevention or musculoskeletal disorders, lower cholesterol and triglyceride levels and reduce the risk of blood clot formation. Omega 3 fatty acids are essential fatty acids that our bodies cannot make by themselves and must be obtained from consumed food. Fish oils, derived from mackerel, lake trout, herring, sardines, albacore tuna and salmon, are also high in two kinds of Omega-3 fatty acids: eicosapentaenoic acid and docosahexaenoic acid.

Omega-3 fatty acids from plant sources include tofu and other forms of soybeans, canola, walnut and flaxseed. Their oils also contain alpha-linolenic acid, another form of omega-3 fatty acid. Omega 3 fatty acids used in the supplement can also be derived from pumpkin seeds, almonds, sesame seeds, walnuts, or combinations of these fatty acids.

From about 1000 mg to about 9000 mg of fiber can be used in the ingestible supplement. Fiber is the elongated, threadlike structures in fruits, vegetables, and grains that cannot be digested. Fiber has long been recognized as one of the best food ingredients for maintaining bowel regularity and preventing constipation.

The two types of fiber are water-soluble and insoluble. Water-soluble fiber dissolves in water and is found in oat bran, legumes, psyllium, nuts, beans, pectins, and various fruits and vegetables. Water-soluble fiber forms a bulky gel in the intestine that regulates the flow of waste materials through the digestive tract. Insoluble fiber cannot be dissolved in water, meaning that our bodies cannot digest it. Insoluble fiber includes the undissolvable parts of plant walls and is found in greatest amounts in cereals, brans, and vegetables. The primary function of insoluble fiber is to collect water that increases stool bulk in the large intestine. Soluble and insoluble fiber can be used alone or in combination.

Other additives can be used in the supplement, such as from 40 mg to 800 mg of a naturally occurring sulfur compound like s-adenosylmethionine. Many biochemical processes in the body are related to s-adenosylmethionine, such as the production and break down of serotonin, melatonin, dopamine, and Vitamin-B12. S-adenosylmethionine also aids in the production of genetic material, such as DNA and cartilage.

Other sulfur components that can be used in the present supplement can be a sulfur compound such as methyl sulfonyl methane, dimethyl sulfoxide and combinations thereof.

The supplement can include minerals, such as from about 1 mg to about 20 mg of selenium, boron, manganese, magnesium and combinations thereof.

In still another embodiment, the supplement can include between from about 1 mg to about 20 mg of a digestive enzyme. Preferred digestive enzymes are bromelain, pepsin, amylase, protease, lipase, cellulase, lactase, alpha-g, glucoamylase, invertase, malt diastase, pectinase, xylanase, bromelain, betain, and trypsin. These digestive enzymes can be in combinations or alone for aiding digestion.

Enzymes help break down food proteins, carbohydrates, and lipids. Mammals digest all their food extra-cellularly. Digestive enzymes are secreted from cells lining the inner surfaces of the exocrine glands. The enzymes hydrolyze the macromolecules in food into small, soluble molecules that can be absorbed into cells.

Additionally, calcium can be included in the supplement in portions from about 250 mg to about 1500 mg. Examples of calcium usable in the supplement are calcium carbonate, calcium citrate, calcium lactate, calcium gluconate and combinations thereof.

The supplement can also include from about 10 mg to about 500 mg of a bioflavonoid. Examples of bioflavonoid are quercetin, grape seed extract and combinations of these bioflavonoids.

Another embodiment relates to a method for improving joint mobility in a subject in need thereof by administering to a person the supplement as described once a day.

The supplement can be prepared by pulverizing the ingredients then blending and passing through an appropriate mesh screen, encapsulating into a capsule form, pressing into tablets, caplets or gelcaps, as well as added to beverages.

Preferably, the supplement is provided to users in dosage quantities ranging from about 2000 mg to about 6000 mg with a preferred dosage of 4089 mg.

A preferred formula of the supplement dosage is as follows:

| Ingredient | Wt/% |
|---|---|
| Glucosamine | 36.2 |
| Cetyl Mysristoleate | 0.96 |
| S-Adenosylmethionine | 0.96 |
| Methylsufonylmethane | 0.96 |
| Digestive Enzymes | 2.28 |
| Spirulina | 4.80 |
| Fatty Acids | 0.24 |
| Vitamin Complex | 53.6 |

Preferably, the vitamin complex in the preferred formula is made of 250 mg of a Vitamin B complex (niacin, calcium pantothenate, pyridoxine hydrochloride (Vitamin-B6), riboflavin (Vitamin-B2), Thiamin Hydrochloride (Vitamin-B1), folic acid, biotin, Vitamin B12); 500 mg of a Vitamin C complex (calcium ascorbate and threonate); 590 mg of a Vitamin E complex (d-alpha tochopheryl or tocopherol); 61.5 mg of a minerals complex (selenium 50 mg, boron 1.5, manganese 10 mg); 300 mg of a Bioflavonoid Complex (quercetin/100 mg and grapeseed extract 200 mg); 15,000 mg of Beta Carotene; and 500 mg of Calcium Lactate.

To manufacture the supplement, the powders are sifted through a fine mesh screen. The spirulina is then added and blended for about 1 minute along with the vitamin complex, the methylsufonylmethane, the glucosamine, the s-adenosylmethionine, and the cetyl mysristoleate. The mixture is passed again through a fine mesh screen and blended for about 3 minutes. The digestive enzymes and fatty acids are added to the mixture and blended for 1 minute. The resultant mixture is then preferably dispensed into three capsules, each weighing about 1,380 mg.

In another embodiment, the ingestible supplement can be prepared as a meal replacement bar.

While this supplement has been described with emphasis on the preferred embodiments, it should be understood that within the scope of the appended claims, the supplement might be practiced other than as specifically described herein.

What is claimed is:

1. A ingestible supplement for treating muskuloskeletal disorders comprising per supplement:
a. from about 250 mg to about 2500 mg of a member selected from the group consisting of 2-amino-2-deoxyglucose sulfate; 2-amino-2-deoxyglucose sulfate hydrochloride; n-acetyl 2-amino-2-deoxyglucose sulfate; and combinations thereof;
b. from about 40 mg to about 750 mg of acetyl myristoleate;
c. from about 40 mg to about 800 mg of s-adenosylmethionine;
d. from about 200 mg to about 2000 mg of a protein;
e. from 100 mg to 2500 mg of a vitamin selected from the group consisting of Vitamin B, Vitamin C, Vitamin E, complexes thereof, and combinations thereof; and
f. from about 1000 mg to about 9000 mg of a fiber.

2. The supplement of claim 1, wherein the supplement comprises from about 1000 mg to about 2000 mg by weight of the member selected from the group consisting of 2-amino-2-deoxyglucose sulfate; 2-amino-2-deoxyglucose sulfate hydrochloride; n-acetyl 2-amino-2-deoxyglucose sulfate; and combinations thereof.

3. The supplement of claim 1, wherein the supplement comprises from about 1200 mg to about 1500 mg by weight of the member selected from the group consisting of 2-amino-2-deoxyglucose sulfate; 2-amino-2-deoxyglucose sulfate hydrochloride; n-acetyl 2-amino-2-deoxyglucose sulfate; and combinations thereof.

4. The supplement of claim 1, wherein the protein is selected from the group consisting of a whey protein, soy protein, sodium casienate protein, legume protein, egg protein, and combinations thereof.

5. The supplement of claim 1, further comprising from about 0.1 mg to about 20 mg of a fatty acid.

6. The supplement of claim 5, wherein the fatty acid is a fatty acid derived from a member of the group consisting of pumpkin seed, almonds, sesame seeds, walnuts, flax seed, soy bean derivatives, and combinations thereof.

7. The supplement of claim 1, wherein the Vitamin B is selected from the group consisting of Vitamin B1, Vitamin B3, Vitamin B5, Vitamin B6, Vitamin B-12, and combinations thereof.

8. The supplement of claim 1, wherein the Vitamin C is selected from the group consisting of ascorbic acid, sodium ascorbate, potassium ascorbate, a calcium ascorbate with at least one threonate, and combinations thereof.

9. The supplement of claim 1, wherein the Vitamin E is selected from the group consisting of tocopherol, tocopheryl tocopheryl acetate, d-alpha tocopherol, d-alpha tocopheryl acetate, d-alpha tocopheryl succinate, complexes thereof, and combinations thereof.

10. The supplement of claim 9, wherein the Vitamin E comprises from about 200 mg to about 1000 mg per supplement.

11. The supplement of claim 1, wherein the fiber is selected from the group consisting of oat bran, a legume, a psyllium, a nut, a bean, pectin, and combinations thereof.

12. The supplement of claim 1, further comprising from about 1 mg to about 20 mg of a mineral selected from the group consisting of selenium, a boron, a manganese, a magnesium, or combinations thereof.

13. The supplement of claim 1, further comprising from about 1 mg to about 20 mg of a digestive enzyme.

14. The supplement of claim 13, wherein the digestive enzyme is selected from the group consisting of bromelain, pepsin, amylase, protease, lipase, cellulase, lactase, alpha-g, glucoamylase, invertase, malt diastase, pectinase, xylanase, bromelain, betain, trypsin, and combinations thereof.

15. The supplement of claim 1, further comprising from about 250 mg to about 1500 mg of a calcium selected from the group consisting of calcium carbonate, calcium citrate, calcium lactate, calcium gluconate, and combinations thereof.

16. The supplement of claim 1, further comprising from about 250 mg to about 1500 mg of Vitamin A.

17. The supplement of claim 16, wherein the Vitamin A is beta carotene.

18. The supplement of claim 16, wherein the Vitamin A is derived from fish oil.

19. The supplement of claim 1, further comprising from about 10 mg to about 500 mg of a bioflavonoid from the group consisting of quercetin, grape seed extract, and combinations thereof.

20. The supplement of claim 1, further comprising from about 250 to about 1000 mg of chondroitin.

21. A method for improving joint mobility in a subject in need thereof comprising the step of administering to the subject an amount of the supplement of claim 1.
